(19) United States
(12) Patent Application Publication
(10) Pub. No.: US 2026/0137388 A1
(43) Pub. Date: May 21, 2026

(54) IMPLANT FOR OCCLUDING A LEFT ATRIAL APPENDAGE

(71) Applicant: BOSTON SCIENTIFIC SCIMED, INC., MAPLE GROVE, MN (US)

(72) Inventors: Philip Andrew Litecky, Forest Lake, MN (US); Troy Anthony Giese, Blaine, MN (US); Patrick Willoughby, Shoreview, MN (US)

(73) Assignee: BOSTON SCIENTIFIC SCIMED, INC., MAPLE GROVE, MN (US)

(21) Appl. No.: 19/392,927

(22) Filed: Nov. 18, 2025

Related U.S. Application Data
(60) Provisional application No. 63/721,843, filed on Nov. 18, 2024.

(51) Int. Cl. A61B 17/12; A61B 17/00

(52) U.S. Cl. CPC A61B 17/12122; A61B 17/12031; A61B 17/12181; A61B 2017/00526

(57) ABSTRACT

An implant includes a plurality of foam layers attached together, wherein each foam layer includes a plurality of wedges monolithically formed with a base portion. A cap layer of foam may be attached at an axial end of the foam layers attached together. The cap layer of foam is disk-shaped and is devoid of wedges. A method of making an implant includes cutting disk-shaped pieces of foam partially through their thickness to define pluralities of wedges monolithically formed with base portions, securing the base portions in a contracted configuration to flare the pluralities of wedges apart from each other, and attaching the base portions such that adjacent pluralities of wedges are interdigitated.

### CROSS-REFERENCE TO RELATED APPLICATIONS

This application claims the benefit of priority of U.S. Provisional Application No. 63/721,843 filed November 18, 2024, the entire disclosure of which is hereby incorporated by reference.

### TECHNICAL FIELD

The disclosure relates generally to medical devices and more particularly to devices, systems, and methods that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices, systems, and methods, each has certain advantages and disadvantages. There is an ongoing need to provide alternative medical devices and systems, and methods for manufacturing and using medical devices and systems.

### SUMMARY

In one example, an implant for occluding a left atrial appendage may comprise a plurality of foam layers attached together, wherein each foam layer of the plurality of foam layers may comprise a plurality of wedges monolithically formed with a base portion.

In addition, or alternatively, to any example described herein, the plurality of wedges of each foam layer are interdigitated with the plurality of wedges of an immediately adjacent foam layer.

In addition, or alternatively, to any example described herein, at least one foam layer of the plurality of foam layers has an outermost radial extent that is different from at least one other foam layer of the plurality of foam layers.

In addition, or alternatively, to any example described herein, each foam layer of the plurality of foam layers has a different outermost radial extent.

In addition, or alternatively, to any example described herein, at least one foam layer of the plurality of foam layers has a density that is different from at least one other foam layer of the plurality of foam layers.

In addition, or alternatively, to any example described herein, at least one wedge of the plurality of wedges within a selected foam layer of the plurality of foam layers has a density that is different from at least one other wedge of the plurality of wedges within the selected foam layer.

In addition, or alternatively, to any example described herein, at least one foam layer of the plurality of foam layers is configured as an anchoring layer configured to exert an anchoring force radially outward against a side wall of the left atrial appendage.

In addition, or alternatively, to any example described herein, the plurality of foam layers comprises a first anchoring layer configured to exert a first anchoring force radially outward against a side wall of the left atrial appendage and a second anchoring layer configured to exert a second anchoring force radially outward against the side wall of the left atrial appendage, wherein the second anchoring layer is axially spaced apart from the first anchoring layer.

In addition, or alternatively, to any example described herein, the plurality of foam layers comprises a low-density fill layer disposed between the first anchoring layer and the second anchoring layer.

In addition, or alternatively, to any example described herein, and in a second example, an implant for occluding a left atrial appendage may comprise a plurality of foam layers attached together, wherein each foam layer of the plurality of foam layers may comprise a plurality of wedges monolithically formed with a base portion, and a cap layer of foam attached at an axial end of the plurality of foam layers attached together. The cap layer of foam may be disk-shaped and may be devoid of wedges.

In addition, or alternatively, to any example described herein, the cap layer of foam has an outermost radial extent that is less than a maximum outermost radial extent of the plurality of foam layers.

In addition, or alternatively, to any example described herein, the cap layer of foam is disposed immediately adjacent a foam layer of the plurality of foam layers that has the maximum outermost radial extent.

In addition, or alternatively, to any example described herein, the cap layer of foam is disposed immediately adjacent a foam layer of the plurality of foam layers having a minimum outermost radial extent of the plurality of foam layers.

In addition, or alternatively, to any example described herein, and in a third example, a method of making an implant for occluding a left atrial appendage may comprise: cutting a first disk-shaped piece of foam partially through a thickness of the first disk-shaped piece of foam to define a first plurality of wedges monolithically formed with a first base portion; securing the first base portion in a contracted configuration to flare the first plurality of wedges apart from each other; cutting a second disk-shaped piece of foam partially through a thickness of the second disk-shaped piece of foam to define a second plurality of wedges monolithically formed with a second base portion; securing the second base portion in a contracted configuration to flare the second plurality of wedges apart from each other; and attaching the first base portion to the second base portion such that the first plurality of wedges is interdigitated with the second plurality of wedges.

In addition, or alternatively, to any example described herein, the first base portion is 25% to 50% of the thickness of the first disk-shaped piece of foam.

In addition, or alternatively, to any example described herein, after attaching the first base portion to the second base portion, each wedge of the first plurality of wedges defines a tip portion and a bottom portion, and each wedge of the second plurality of wedges defines a tip portion and a bottom portion.

In addition, or alternatively, to any example described herein, the tip portion of the first plurality of wedges extends in a first direction from the bottom portion of the first plurality of wedges, and the tip portion of the second plurality of wedges extends in the first direction from the bottom portion of the second plurality of wedges.

In addition, or alternatively, to any example described herein, cutting the first disk-shaped piece of foam partially through the thickness of the first disk-shaped piece of foam comprises cutting downward into a top surface of the first disk-shaped piece of foam.

In addition, or alternatively, to any example described herein, after securing the first base portion in the contracted configuration, the top surface of the first disk-shaped piece of foam faces radially outwardly.

In addition, or alternatively, to any example described herein, the method may comprise attaching a cap layer of foam to the first disk-shaped piece of foam opposite the second disk-shaped piece of foam, wherein an outermost radial extent of the cap layer of foam is less than an outermost radial extent of the first disk-shaped piece of foam.

In addition, or alternatively, to any example described herein, method may comprise: cutting a third disk-shaped piece of foam partially through a thickness of the third disk-shaped piece of foam to define a third plurality of wedges monolithically formed with a third base portion; securing the third base portion in a contracted configuration to flare the third plurality of wedges apart from each other; and attaching the third base portion to the second base portion such that the second plurality of wedges is interdigitated with the third plurality of wedges.

In addition, or alternatively, to any example described herein, the method may comprise: cutting a fourth disk-shaped piece of foam partially through a thickness of the fourth disk-shaped piece of foam to define a fourth plurality of wedges monolithically formed with a fourth base portion; securing the fourth base portion in a contracted configuration to flare the fourth plurality of wedges apart from each other; and attaching the fourth base portion to the third base portion such that the third plurality of wedges is interdigitated with the fourth plurality of wedges.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-3 illustrate selected aspects of an implant for occluding a left atrial appendage;

FIGS. 4-12 illustrate selected aspects of a method of making the implant; and

FIG. 13 illustrates selected aspects related to deploying the implant in the left atrial appendage.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the disclosure. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The devices, systems, and/or methods disclosed herein may provide a number of desirable features and benefits as described in more detail below.

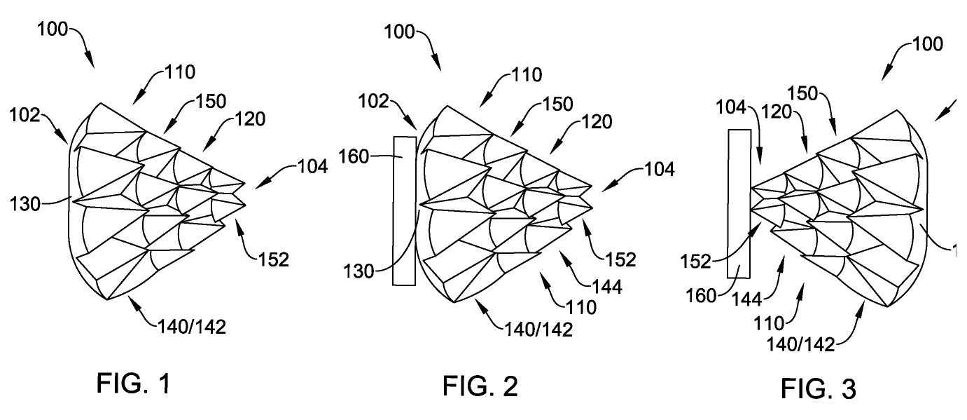

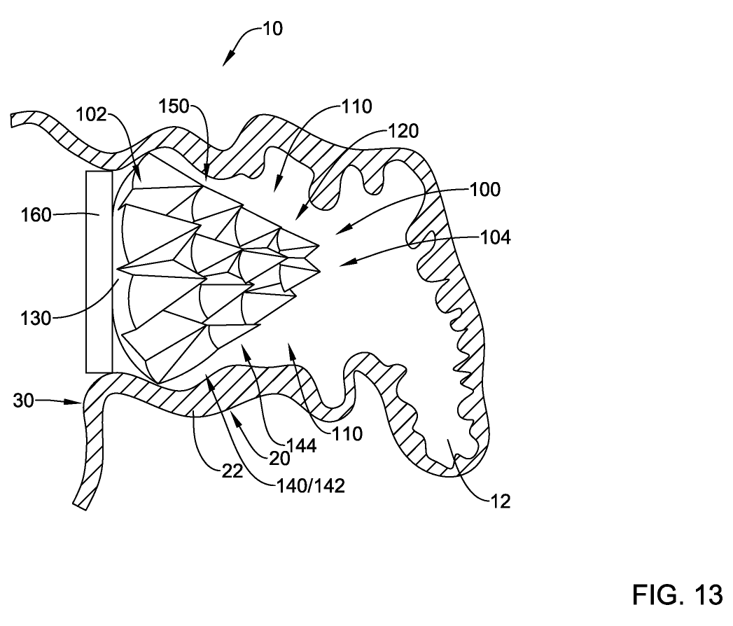

FIGS. 1-3 illustrate selected aspects of an implant 100 for occluding a left atrial appendage (e.g., FIG. 13). In some embodiments, the implant 100 may comprise a plurality of foam layers 110 attached together. In some embodiments, the plurality of foam layers 110 may extend from a first axial end 102 of the implant 100 to a second axial end 104 of the implant 100. In some embodiments, the first axial end 102 of the implant 100 may be a proximal end of the implant 100 and the second axial end 104 of the implant 100 may be a distal end of the implant 100. In some embodiments, the first axial end 102 of the implant 100 may be a distal end of the implant 100 and the second axial end 104 of the implant 100 may be a proximal end of the implant 100.

The implant 100 and/or the plurality of foam layers 110 may be configured to shift from a collapsed configuration to an expanded configuration. The implant 100 and/or the plurality of foam layers 110 may be disposed in the collapsed configuration during delivery. In some embodiments, the implant 100 and/or the plurality of foam layers 110 may be constrained within a delivery device. In some embodiments, the implant 100 and/or the plurality of foam layers 110 may be self-constrained in the collapsed configuration. Other configurations are also contemplated. The implant 100 and/or the plurality of foam layers 110 may be configured to shift toward and/or to the expanded configuration after delivery and/or after being deployed within the left atrial appendage. In some embodiments, the implant 100 and/or the plurality of foam layers 110 may be configured to shift toward and/or to the expanded configuration when unconstrained. In some embodiments, the implant 100 and/or the plurality of foam layers 110 may be configured to shift toward and/or to the expanded configuration upon exposure to a predetermined stimulus (e.g., fluid, temperature, etc.). Other configurations are also contemplated.

In some embodiments, each foam layer of the plurality of foam layers 110 may comprise a plurality of wedges 120 monolithically formed with a base portion 130. In some embodiments, the base portion 130 and the plurality of wedges 120 of each foam layer of the plurality of foam layers 110 may be formed from a single monolithic piece of foam, as discussed herein. Other configurations are also contemplated.

In some embodiments, the plurality of wedges 120 of each foam layer of the plurality of foam layers 110 may be interdigitated with the plurality of wedges 120 of an immediately adjacent foam layer of the plurality of foam layers 110. For example, at least a portion of an individual wedge of a first foam layer may extend between two immediately adjacent wedges of a second foam layer disposed immediately adjacent the first foam layer. In some embodiments, the plurality of wedges 120 of each foam layer of the plurality of foam layers 110 may be circumferentially offset from the plurality of wedges 120 of each immediately adjacent foam layer of the plurality of foam layers 110. Other configurations are also contemplated.

In some embodiments, having the plurality of wedges 120 of adjacent foam layers offset from and/or interdigitated with each other may permit the plurality of wedges 120 of each foam layer to collapse and crimp down into free space between the plurality of wedges 120 of each adjacent foam layer, thereby permitting the collapsed configuration to be smaller for delivery than if the plurality of wedges 120 of adjacent foam layers are aligned with each other. Additionally, having the plurality of wedges 120 of adjacent foam layers offset from and/or interdigitated with each other may reduce fluid leak pathways through implant 100.

In some embodiments, at least one foam layer of the plurality of foam layers 110 may have an outermost radial extent that is different from at least one other foam layer of the plurality of foam layers 110. In some embodiments, each foam layer of the plurality of foam layers 110 may have a different outermost radial extent. In some embodiments, each foam layer of the plurality of foam layers 110 may have an outermost radial extent that is different from an outermost radial extent of every other foam layer of the plurality of foam layers 110. Other configurations are also contemplated.

In some embodiments, at least one foam layer of the plurality of foam layers 110 may have a density that is different from at least one other foam layer of the plurality of foam layers 110. In some embodiments, each foam layer of the plurality of foam layers 110 may have a different density. In some embodiments, the plurality of foam layers 110 may have alternating densities along a central longitudinal axis of the implant 100. In one non-limiting example, the plurality of foam layers 110 may comprise a first foam layer having a first density, a second foam layer disposed immediately adjacent to the first foam layer and having a second density different from the first density, a third foam layer disposed immediately adjacent to the second foam layer and having a third density different from the first density and/or the second density, etc. In some embodiments, each foam layer of the plurality of foam layers 110 may have a density that is substantially identical to every other foam layer of the plurality of foam layers 110. In some embodiments, at least one wedge of the plurality of wedges 120 within a selected foam layer of the plurality of foam layers 110 may have a density that is different from at least one other wedge of the plurality of wedges 120 within the selected foam layer of the plurality of foam layers 110. For example, in some embodiments, multiple and/or different densities may exist within a single foam layer of the plurality of foam layers 110. Other configurations are also contemplated. Higher density foam may create and/or cause increased stiffness, which may improve anchoring and/or fixation of the foam and/or the implant 100 within the left atrial appendage. Lower density foam may provide a high collapsibility ratio and/or a greater degree of volume change when expanding, thereby permitting the lower density foam to “puff out” when it expands, which may be beneficial for filling in spaces and/or gaps within the implant 100 and/or the left atrial appendage to reduce and/or fill leak pathways around and/or through the implant 100.

In some embodiments, at least one foam layer of the plurality of foam layers 110 may optionally be configured as an anchoring layer 140 configured to exert an anchoring force radially outward against a side wall of the left atrial appendage when the implant 100 is disposed and/or unconstrained within the left atrial appendage. In some embodiments, the plurality of foam layers 110 may optionally comprise a first anchoring layer 142 configured to exert a first anchoring force radially outward against the side wall of the left atrial appendage when the implant 100 is disposed and/or unconstrained within the left atrial appendage, and a second anchoring layer 144 configured to exert a second anchoring force radially outward against the side wall of the left atrial appendage when the implant 100 is disposed and/or unconstrained within the left atrial appendage. In some embodiments, the first anchoring layer 142 may be disposed proximate and/or at the first axial end 102 of the implant 100. In at least some embodiments, the second anchoring layer 144 may be axially spaced apart from the first anchoring layer 142. In some embodiments, the plurality of foam layers 110 may optionally comprise a low-density fill layer 150 disposed between the first anchoring layer 142 and the second anchoring layer 144. The low-density fill layer 150 may be configured to fill space within the left atrial appendage while avoiding applying radially outward force against the side wall of the left atrial appendage. In some embodiments, the plurality of foam layers 110 may optionally comprise a second low-density fill layer 152 disposed proximate and/or at the second axial end 104 of the implant 100. Other configurations and/or arrangements are also contemplated.

In some embodiments, the implant 100 may comprise a cap layer of foam 160 attached at an axial end (e.g., the first axial end 102, the second axial end 104, the proximal end, the distal end, etc.) of the plurality of foam layers 110 attached together, as seen in FIGS. 2-3. In some embodiments, the cap layer of foam 160 may be configured to fill, obstruct, and/or close off the ostium of the left atrial appendage when unconstrained and/or when disposed in an expanded configuration. In some embodiments, the cap layer of foam 160 may be disk-shaped when unconstrained and/or when disposed in the expanded configuration. In some embodiments, the cap layer of foam 160 may be oblong, oval, or egg shaped. Other configurations are also contemplated. In at least some embodiments, the cap layer of foam 160 may be devoid of wedges (e.g., features formed like the plurality of wedges 120 discussed herein). In some embodiments, the cap layer of foam 160 may be devoid of recesses, holes, apertures, cutouts, etc. formed in the cap layer of foam 160. For the purpose of this disclosure, the recesses, holes, apertures, cutouts, etc. formed in the cap layer of foam 160 shall be understood to be features that are not inherent to a normal foam structure, which may be or include open-celled foam.

In some embodiments, the cap layer of foam 160 may have an outermost radial extent that is less than a maximum outermost radial extent (e.g., the greatest outermost radial extent) of the plurality of foam layers 110. In some embodiments, the cap layer of foam 160 may be disposed immediately adjacent a foam layer of the plurality of foam layers 110 that has the maximum outermost radial extent (e.g., the greatest outermost radial extent) of the plurality of foam layers 110. In some embodiments, the cap layer of foam 160 may be disposed immediately adjacent a foam layer of the plurality of foam layers 110 that has a minimum outermost radial extent (e.g., the smallest outermost radial extent) of the plurality of foam layers 110. In some embodiments, the cap layer of foam 160 may define a proximalmost surface of the implant 100. In some embodiments, the proximalmost surface of the implant 100 may be configured to face outward from the left atrial appendage and/or to face toward the left atrium. Other configurations are also contemplated.

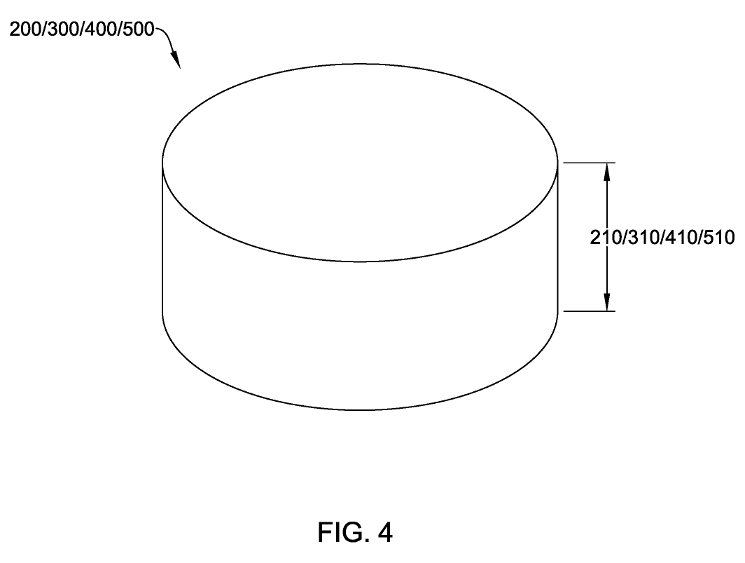

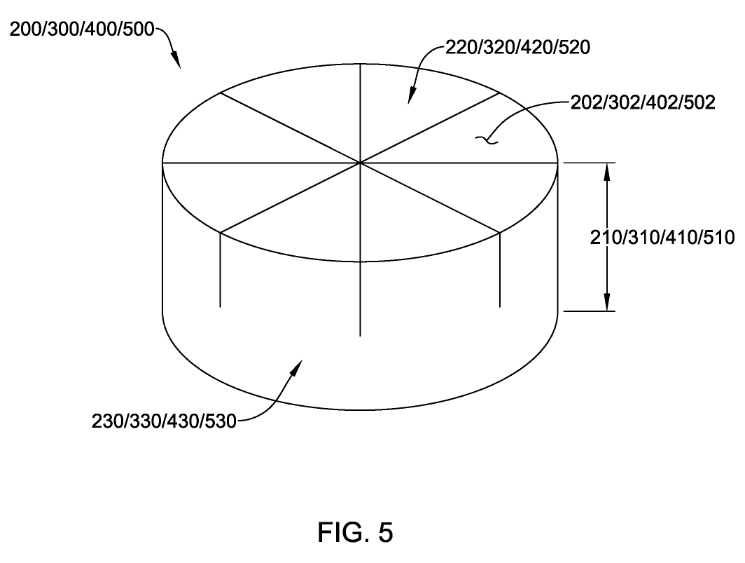

FIGS. 4-12 illustrate selected aspects of methods of making the implant 100 for occluding the left atrial appendage. In some embodiments, a method of making the implant 100 for occluding the left atrial appendage may comprise cutting a first disk-shaped piece of foam 200 partially through a thickness 210 of the first disk-shaped piece of foam 200 to define a first plurality of wedges 220 monolithically formed with a first base portion 230, as seen in FIG. 5. When making the implant 100, each foam layer and/or the elements thereof start as a single monolithic piece of foam (e.g., the first disk-shaped piece of foam 200, etc.), as seen in FIG. 4. This requires fewer steps than assembling multiple pieces to form each foam layer, while avoiding degradation, distortion, pore clogging, etc. that may occur when bonding individual pieces of foam together.

In some embodiments, cutting the first disk-shaped piece of foam 200 partially through the thickness 210 of the first disk-shaped piece of foam 200 may comprise cutting downward into a top surface 202 of the first disk-shaped piece of foam 200. In some embodiments, cutting the first disk-shaped piece of foam 200 partially through the thickness 210 of the first disk-shaped piece of foam 200 may comprise cutting the first disk-shaped piece of foam 200 with a blade (e.g., a razor blade, a knife, a scalpel, etc.). Other means for cutting the first disk-shaped piece of foam 200, including but not limited to a laser, a wire, etc., are also contemplated. In some embodiments, the first base portion 230 may be between about 20% and about 60% of the thickness 210 of the first disk-shaped piece of foam 200. In some embodiments, the first base portion 230 may be between about 25% and about 50% of the thickness 210 of the first disk-shaped piece of foam 200. In some embodiments, the first base portion 230 may be between about 30% and about 40% of the thickness 210 of the first disk-shaped piece of foam 200. Other configurations are also contemplated.

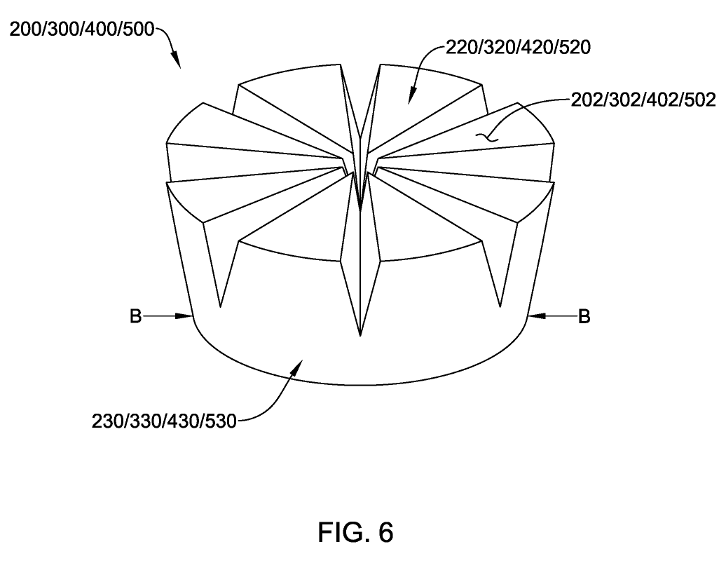

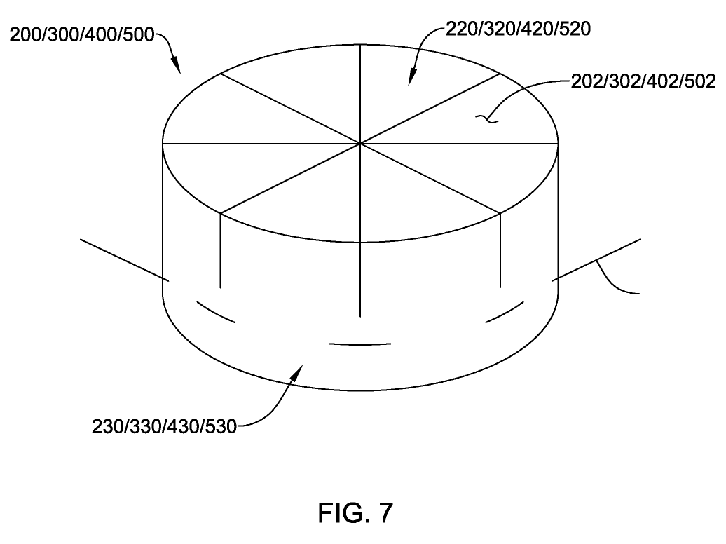

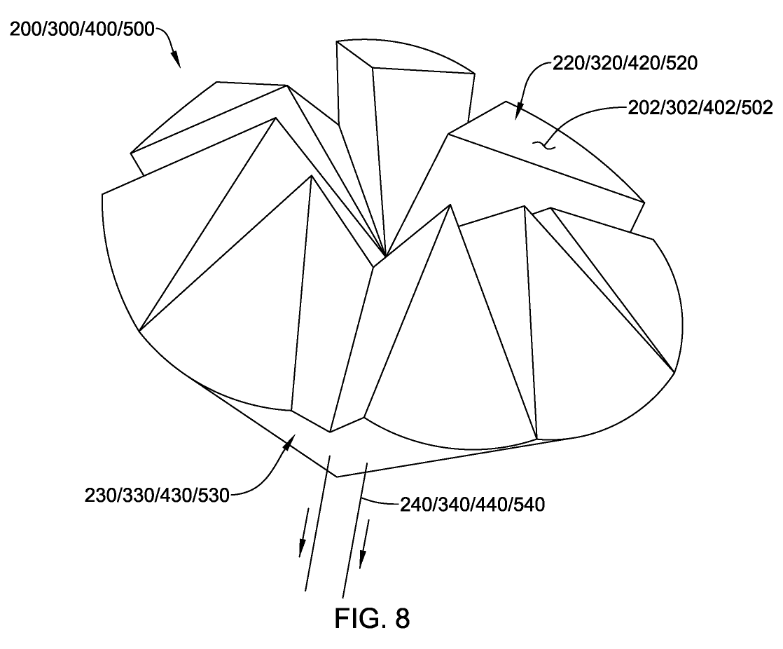

In some embodiments, the method of making the implant 100 for occluding the left atrial appendage may comprise securing the first base portion 230 in a contracted configuration to flare the first plurality of wedges 220 apart from each other, as seen in FIGS. 6-9. In some embodiments, securing the first base portion 230 in the contracted configuration may comprise pinching or squeezing the first base portion 230 to radially collapse the first base portion 230 (in the direction of the arrows “B” indicated near the first base portion 230 in FIG. 6), thereby flaring the first plurality of wedges 220 apart from each other, as seen in FIG. 6. In some embodiments, securing the first base portion 230 in the contracted configuration may comprise disposing a first suture 240 around at least a portion of the first base portion 230, as seen in FIG. 7. In some embodiments, disposing the first suture 240 around at least a portion of the first base portion 230 may comprise stitching and/or interweaving the first suture 240 with the first base portion 230. In some embodiments, securing the first base portion 230 in the contracted configuration may comprise thereafter tightening the first suture 240, thereby radially collapsing the first base portion 230 toward and/or to the contracted configuration, as seen in FIG. 8. In some alternative embodiments, the first base portion 230 may be secured in the contracted configuration by and/or with a crimp ring, or other tubular element. Other configurations are also contemplated.

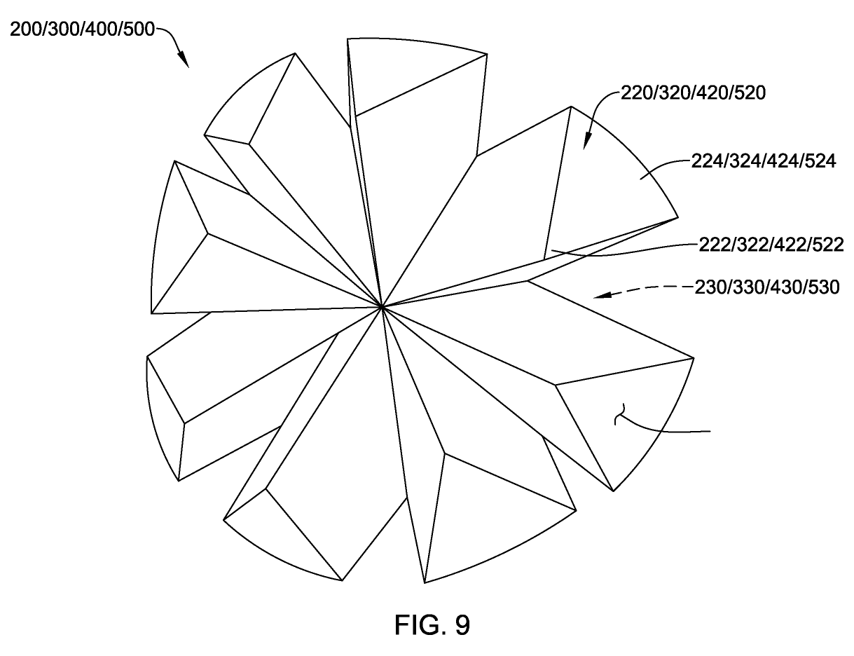

In some embodiments, after securing the first base portion 230 in the contracted configuration, the top surface 202 of the first disk-shaped piece of foam 200 may face radially outwardly, as seen in FIG. 9, instead of facing upwards. Accordingly, securing the first base portion 230 in the contracted configuration, such as by tightening the first suture 240, causes the first plurality of wedges 220 to flare outwardly such that the top surface 202 of the first disk-shaped piece of foam 200 shifts from facing upwardly to facing radially outwardly. In some embodiments, prior to securing the first base portion 230 in the contracted configuration, the top surface 202 of the first disk-shaped piece of foam 200 may face upwardly and/or may only face upwardly, and after securing the first base portion 230 in the contracted configuration, the top surface 202 of the first disk-shaped piece of foam 200 may face radially outwardly and/or may face only radially outwardly. In some embodiments, prior to securing the first base portion 230 in the contracted configuration, the top surface 202 of the first disk-shaped piece of foam 200 may face upwardly and/or may only face upwardly, and after securing the first base portion 230 in the contracted configuration, the top surface 202 of the first disk-shaped piece of foam 200 may face partially upwardly and partially radially outwardly (e.g., the top surface 202 may be angled to face away from a central longitudinal axis of the first disk-shaped piece of foam 200). Other configurations are also contemplated.

In some embodiments, after securing the first base portion 230 in the contracted configuration, each wedge of the first plurality of wedges 220 may define a tip portion 222 and a bottom portion 224. In some embodiments, after securing the first base portion 230 in the contracted configuration, the tip portion 222 of each wedge of the first plurality of wedges 220 may extend in a first direction from the bottom portion 224 of each wedge of the first plurality of wedges 220. In some embodiments, the first direction may be generally parallel to the central longitudinal axis of the first disk-shaped piece of foam 200. Other configurations are also contemplated.

In some embodiments, the method of making the implant 100 for occluding the left atrial appendage may comprise cutting a second disk-shaped piece of foam 300 partially through a thickness 310 of the second disk-shaped piece of foam 300 to define a second plurality of wedges 320 monolithically formed with a second base portion 330, as seen in FIG. 5. In at least some embodiments, the second disk-shaped piece of foam 300 may have an outermost radial extent that is different from an outermost radial extent of the first disk-shaped piece of foam 200. In some embodiments, the second disk-shaped piece of foam 300 may have an outermost radial extent that is less than an outermost radial extent of the first disk-shaped piece of foam 200. In some embodiments, the second disk-shaped piece of foam 300 may have an outermost radial extent that is greater than an outermost radial extent of the first disk-shaped piece of foam 200.

In some embodiments, cutting the second disk-shaped piece of foam 300 partially through the thickness 310 of the second disk-shaped piece of foam 300 may comprise cutting downward into a top surface 302 of the second disk-shaped piece of foam 300. In some embodiments, cutting the second disk-shaped piece of foam 300 partially through the thickness 310 of the second disk-shaped piece of foam 300 may comprise cutting the second disk-shaped piece of foam 300 with a blade (e.g., a razor blade, a knife, a scalpel, etc.). Other means for cutting the second disk-shaped piece of foam 300, including but not limited to a laser, a wire, etc., are also contemplated. In some embodiments, the second base portion 330 may be between about 20% and about 60% of the thickness 310 of the second disk-shaped piece of foam 300. In some embodiments, the second base portion 330 may be between about 25% and about 50% of the thickness 310 of the second disk-shaped piece of foam 300. In some embodiments, the second base portion 330 may be between about 30% and about 40% of the thickness 310 of the second disk-shaped piece of foam 300. Other configurations are also contemplated.

In some embodiments, the method of making the implant 100 for occluding the left atrial appendage may comprise securing the second base portion 330 in a contracted configuration to flare the second plurality of wedges 320 apart from each other, as seen in FIGS. 6-9. In some embodiments, securing the second base portion 330 in the contracted configuration may comprise pinching or squeezing the second base portion 330 to radially collapse the second base portion 330 (in the direction of the arrows “B” indicated near the second base portion 330 in FIG. 6), thereby flaring the second plurality of wedges 320 apart from each other, as seen in FIG. 6. In some embodiments, securing the second base portion 330 in the contracted configuration may comprise disposing a second suture 340 around at least a portion of the second base portion 330, as seen in FIG. 7. In some embodiments, disposing the second suture 340 around at least a portion of the second base portion 330 may comprise stitching and/or interweaving the second suture 340 with the second base portion 330. In some embodiments, securing the second base portion 330 in the contracted configuration may comprise thereafter tightening the second suture 340, thereby radially collapsing the second base portion 330 toward and/or to the contracted configuration, as seen in FIG. 8. In some alternative embodiments, the second base portion 330 may be secured in the contracted configuration by and/or with a crimp ring, or other tubular element. Other configurations are also contemplated.

In some embodiments, after securing the second base portion 330 in the contracted configuration, the top surface 302 of the second disk-shaped piece of foam 300 may face radially outwardly, as seen in FIG. 9, instead of facing upwards. Accordingly, securing the second base portion 330 in the contracted configuration, such as by tightening the second suture 340, causes the second plurality of wedges 320 to flare outwardly such that the top surface 302 of the second disk-shaped piece of foam 300 shifts from facing upwardly to facing radially outwardly. In some embodiments, prior to securing the second base portion 330 in the contracted configuration, the top surface 302 of the second disk-shaped piece of foam 300 may face upwardly and/or may only face upwardly, and after securing the second base portion 330 in the contracted configuration, the top surface 302 of the second disk-shaped piece of foam 300 may face radially outwardly and/or may face only radially outwardly. In some embodiments, prior to securing the second base portion 330 in the contracted configuration, the top surface 302 of the second disk-shaped piece of foam 300 may face upwardly and/or may only face upwardly, and after securing the second base portion 330 in the contracted configuration, the top surface 302 of the second disk-shaped piece of foam 300 may face partially upwardly and partially radially outwardly (e.g., the top surface 302 may be angled to face away from a central longitudinal axis of the second disk-shaped piece of foam 300). Other configurations are also contemplated.

In some embodiments, after securing the second base portion 330 in the contracted configuration, each wedge of the second plurality of wedges 320 may define a tip portion 322 and a bottom portion 324. In some embodiments, after securing the second base portion 330 in the contracted configuration the tip portion 322 of each wedge of the second plurality of wedges 320 may extend in the first direction from the bottom portion 324 of each wedge of the second plurality of wedges 320.

In some embodiments, the method of making the implant 100 for occluding the left atrial appendage may comprise attaching the first base portion 230 of the first disk-shaped piece of foam 200 to the second base portion 330 of the second disk-shaped piece of foam 300 such that the first plurality of wedges 220 of the first disk-shaped piece of foam 200 is interdigitated with the second plurality of wedges 320 of the second disk-shaped piece of foam 300, as seen schematically in FIGS. 1-3. In some embodiments, after attaching the first base portion 230 to the second base portion 330, the tip portion 222 of the first plurality of wedges 220 extends in the first direction from the bottom portion 224 of the first plurality of wedges 220, and the tip portion 322 of the second plurality of wedges 320 extends in the first direction from the bottom portion 324 of the second plurality of wedges 320. In some embodiments, the tip portion 222 of the first plurality of wedges 220 extends in the same general direction from the bottom portion 224 of the first plurality of wedges 220 as the tip portion 322 of the second plurality of wedges 320 extends from the bottom portion 324 of the second plurality of wedges 320. Other configurations are also contemplated.

In some embodiments, the method of making the implant 100 for occluding the left atrial appendage may comprise cutting a third disk-shaped piece of foam 400 partially through a thickness 410 of the third disk-shaped piece of foam 400 to define a third plurality of wedges 420 monolithically formed with a third base portion 430, as seen in FIG. 5. In at least some embodiments, the third disk-shaped piece of foam 400 may have an outermost radial extent that is different from an outermost radial extent of the first disk-shaped piece of foam 200 and/or the second disk-shaped piece of foam 300. In some embodiments, the third disk-shaped piece of foam 400 may have an outermost radial extent that is less than an outermost radial extent of the first disk-shaped piece of foam 200 and/or the second disk-shaped piece of foam 300. In some embodiments, the third disk-shaped piece of foam 400 may have an outermost radial extent that is greater than an outermost radial extent of the first disk-shaped piece of foam 200 and/or the second disk-shaped piece of foam 300.

In some embodiments, cutting the third disk-shaped piece of foam 400 partially through the thickness 410 of the third disk-shaped piece of foam 400 may comprise cutting downward into a top surface 402 of the third disk-shaped piece of foam 400. In some embodiments, cutting the third disk-shaped piece of foam 400 partially through the thickness 410 of the third disk-shaped piece of foam 400 may comprise cutting the third disk-shaped piece of foam 400 with a blade (e.g., a razor blade, a knife, a scalpel, etc.). Other means for cutting the third disk-shaped piece of foam 400, including but not limited to a laser, a wire, etc., are also contemplated. In some embodiments, the third base portion 430 may be between about 20% and about 60% of the thickness 410 of the third disk-shaped piece of foam 400. In some embodiments, the third base portion 430 may be between about 25% and about 50% of the thickness 410 of the third disk-shaped piece of foam 400. In some embodiments, the third base portion 430 may be between about 30% and about 40% of the thickness 410 of the third disk-shaped piece of foam 400. Other configurations are also contemplated.

In some embodiments, the method of making the implant 100 for occluding the left atrial appendage may comprise securing the third base portion 430 in a contracted configuration to flare the third plurality of wedges 420 apart from each other, as seen in FIGS. 6-8. In some embodiments, securing the third base portion 430 in the contracted configuration may comprise pinching or squeezing the third base portion 430 to radially collapse the third base portion 430 (in the direction of the arrows “B” indicated near the third base portion 430 in FIG. 6), thereby flaring the third plurality of wedges 420 apart from each other, as seen in FIG. 6. In some embodiments, securing the third base portion 430 in the contracted configuration may comprise disposing a third suture 440 around at least a portion of the third base portion 430, as seen in FIG. 7. In some embodiments, disposing the third suture 440 around at least a portion of the third base portion 430 may comprise stitching and/or interweaving the third suture 440 with the third base portion 430. In some embodiments, securing the third base portion 430 in the contracted configuration may comprise thereafter tightening the third suture 440, thereby radially collapsing the third base portion 430 toward and/or to the contracted configuration, as seen in FIG. 8. In some alternative embodiments, the third base portion 430 may be secured in the contracted configuration by and/or with a crimp ring, or other tubular element. Other configurations are also contemplated.

In some embodiments, after securing the third base portion 430 in the contracted configuration, the top surface 402 of the third disk-shaped piece of foam 400 may face radially outwardly, as seen in FIG. 9, instead of facing upwards. Accordingly, securing the third base portion 430 in the contracted configuration, such as by tightening the third suture 440, causes the third plurality of wedges 420 to flare outwardly such that the top surface 402 of the third disk-shaped piece of foam 400 shifts from facing upwardly to facing radially outwardly. In some embodiments, prior to securing the third base portion 430 in the contracted configuration, the top surface 402 of the third disk-shaped piece of foam 400 may face upwardly and/or may only face upwardly, and after securing the third base portion 430 in the contracted configuration, the top surface 402 of the third disk-shaped piece of foam 400 may face radially outwardly and/or may face only radially outwardly. In some embodiments, prior to securing the third base portion 430 in the contracted configuration, the top surface 402 of the third disk-shaped piece of foam 400 may face upwardly and/or may only face upwardly, and after securing the third base portion 430 in the contracted configuration, the top surface 402 of the third disk-shaped piece of foam 400 may face partially upwardly and partially radially outwardly (e.g., the top surface 402 may be angled to face away from a central longitudinal axis of the third disk-shaped piece of foam 400). Other configurations are also contemplated.

In some embodiments, after securing the third base portion 430 in the contracted configuration, each wedge of the third plurality of wedges 420 may define a tip portion 422 and a bottom portion 424. In some embodiments, after securing the third base portion 430 in the contracted configuration the tip portion 422 of each wedge of the third plurality of wedges 420 may extend in the first direction from the bottom portion 424 of each wedge of the third plurality of wedges 420.

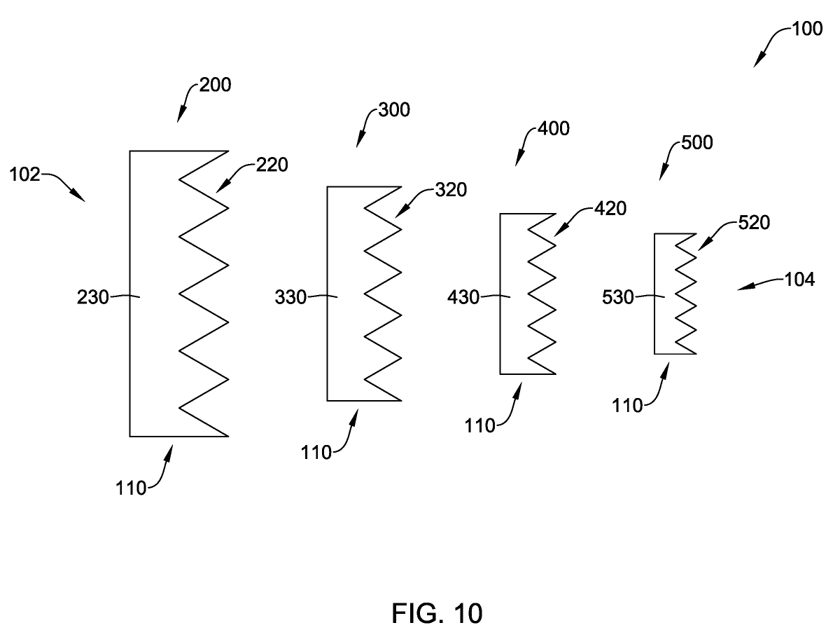

In some embodiments, the method of making the implant 100 for occluding the left atrial appendage may comprise attaching the third base portion 430 of the third disk-shaped piece of foam 400 to the second base portion 330 of the second disk-shaped piece of foam 300, as seen in FIGS. 10-12. In some embodiments, the method of making the implant 100 for occluding the left atrial appendage may comprise attaching the third base portion 430 of the third disk-shaped piece of foam 400 to the second base portion 330 of the second disk-shaped piece of foam 300 such that the second plurality of wedges 320 of the second disk-shaped piece of foam 300 is interdigitated with the third plurality of wedges 420 of the third disk-shaped piece of foam 400. In some embodiments, after attaching the third base portion 430 to the second base portion 330, the tip portion 222 of the first plurality of wedges 220 extends in the first direction from the bottom portion 224 of the first plurality of wedges 220, and the tip portion 322 of the second plurality of wedges 320 extends in the first direction from the bottom portion 324 of the second plurality of wedges 320, and the tip portion 422 of the third plurality of wedges 420 extends in the first direction from the bottom portion 424 of the third plurality of wedges 420. In some embodiments, the tip portion 422 of the third plurality of wedges 420 extends in the same general direction from the bottom portion 424 of the third plurality of wedges 420 as the tip portion 322 of the second plurality of wedges 320 extends from the bottom portion 324 of the second plurality of wedges 320 and/or the tip portion 222 of the first plurality of wedges 220 extends from the bottom portion 224 of the first plurality of wedges 220. Other configurations are also contemplated.

In some embodiments, the method of making the implant 100 for occluding the left atrial appendage may comprise cutting a fourth disk-shaped piece of foam 500 partially through a thickness 510 of the fourth disk-shaped piece of foam 500 to define a fourth plurality of wedges 520 monolithically formed with a fourth base portion 530, as seen in FIG. 5. In at least some embodiments, the fourth disk-shaped piece of foam 500 may have an outermost radial extent that is different from an outermost radial extent of the first disk-shaped piece of foam 200, the second disk-shaped piece of foam 300, and/or the third disk-shaped piece of foam 400. In some embodiments, the fourth disk-shaped piece of foam 500 may have an outermost radial extent that is less than an outermost radial extent of the first disk-shaped piece of foam 200, the second disk-shaped piece of foam 300, and/or the third disk-shaped piece of foam 400. In some embodiments, the fourth disk-shaped piece of foam 500 may have an outermost radial extent that is greater than an outermost radial extent of the first disk-shaped piece of foam 200, the second disk-shaped piece of foam 300, and/or the third disk-shaped piece of foam 400.

In some embodiments, cutting the fourth disk-shaped piece of foam 500 partially through the thickness 510 of the fourth disk-shaped piece of foam 500 may comprise cutting downward into a top surface 502 of the fourth disk-shaped piece of foam 500. In some embodiments, cutting the fourth disk-shaped piece of foam 500 partially through the thickness 510 of the fourth disk-shaped piece of foam 500 may comprise cutting the fourth disk-shaped piece of foam 500 with a blade (e.g., a razor blade, a knife, a scalpel, etc.). Other means for cutting the fourth disk-shaped piece of foam 500, including but not limited to a laser, a wire, etc., are also contemplated. In some embodiments, the fourth base portion 530 may be between about 20% and about 60% of the thickness 510 of the fourth disk-shaped piece of foam 500. In some embodiments, the fourth base portion 530 may be between about 25% and about 50% of the thickness 510 of the fourth disk-shaped piece of foam 500. In some embodiments, the fourth base portion 530 may be between about 30% and about 40% of the thickness 510 of the fourth disk-shaped piece of foam 500. Other configurations are also contemplated.

In some embodiments, the method of making the implant 100 for occluding the left atrial appendage may comprise securing the fourth base portion 530 in a contracted configuration to flare the fourth plurality of wedges 420 apart from each other, as seen in FIGS. 6-8. In some embodiments, securing the fourth base portion 530 in the contracted configuration may comprise pinching or squeezing the fourth base portion 530 to radially collapse the fourth base portion 530 (in the direction of the arrows “B” indicated near the fourth base portion 430 in FIG. 6), thereby flaring the fourth plurality of wedges 520 apart from each other, as seen in FIG. 6. In some embodiments, securing the fourth base portion 530 in the contracted configuration may comprise disposing a fourth suture 540 around at least a portion of the fourth base portion 530, as seen in FIG. 7. In some embodiments, disposing the fourth suture 540 around at least a portion of the fourth base portion 530 may comprise stitching and/or interweaving the fourth suture 540 with the fourth base portion 530. In some embodiments, securing the fourth base portion 530 in the contracted configuration may comprise thereafter tightening the fourth suture 540, thereby radially collapsing the fourth base portion 530 toward and/or to the contracted configuration, as seen in FIG. 8. In some alternative embodiments, the fourth base portion 530 may be secured in the contracted configuration by and/or with a crimp ring, or other tubular element. Other configurations are also contemplated.

In some embodiments, after securing the fourth base portion 530 in the contracted configuration, the top surface 502 of the fourth disk-shaped piece of foam 500 may face radially outwardly, as seen in FIG. 9, instead of facing upwards. Accordingly, securing the fourth base portion 530 in the contracted configuration, such as by tightening the fourth suture 540, causes the fourth plurality of wedges 520 to flare outwardly such that the top surface 502 of the fourth disk-shaped piece of foam 500 shifts from facing upwardly to facing radially outwardly. In some embodiments, prior to securing the fourth base portion 530 in the contracted configuration, the top surface 502 of the fourth disk-shaped piece of foam 500 may face upwardly and/or may only face upwardly, and after securing the fourth base portion 530 in the contracted configuration, the top surface 502 of the fourth disk-shaped piece of foam 500 may face radially outwardly and/or may face only radially outwardly. In some embodiments, prior to securing the fourth base portion 530 in the contracted configuration, the top surface 502 of the fourth disk-shaped piece of foam 500 may face upwardly and/or may only face upwardly, and after securing the fourth base portion 530 in the contracted configuration, the top surface 502 of the fourth disk-shaped piece of foam 500 may face partially upwardly and partially radially outwardly (e.g., the top surface 502 may be angled to face away from a central longitudinal axis of the fourth disk-shaped piece of foam 500). Other configurations are also contemplated.

In some embodiments, after securing the fourth base portion 530 in the contracted configuration, each wedge of the fourth plurality of wedges 520 may define a tip portion 522 and a bottom portion 524. In some embodiments, after securing the fourth base portion 530 in the contracted configuration the tip portion 522 of each wedge of the fourth plurality of wedges 520 may extend in the first direction from the bottom portion 524 of each wedge of the fourth plurality of wedges 520.

In some embodiments, the method of making the implant 100 for occluding the left atrial appendage may comprise attaching the fourth base portion 530 of the fourth disk-shaped piece of foam 500 to the third base portion 430 of the third disk-shaped piece of foam 400, as seen in FIGS. 10-12. In some embodiments, the method of making the implant 100 for occluding the left atrial appendage may comprise attaching the fourth base portion 530 of the fourth disk-shaped piece of foam 500 to the third base portion 430 of the third disk-shaped piece of foam 400 such that the third plurality of wedges 420 of the third disk-shaped piece of foam 400 is interdigitated with the fourth plurality of wedges 520 of the fourth disk-shaped piece of foam 500. In some embodiments, after attaching the third base portion 430 to the second base portion 330, the tip portion 222 of the first plurality of wedges 220 extends in the first direction from the bottom portion 224 of the first plurality of wedges 220, and the tip portion 322 of the second plurality of wedges 320 extends in the first direction from the bottom portion 324 of the second plurality of wedges 320, and the tip portion 422 of the third plurality of wedges 420 extends in the first direction from the bottom portion 424 of the third plurality of wedges 420, and the tip portion 522 of the fourth plurality of wedges 520 extends in the first direction from the bottom portion 524 of the fourth plurality of wedges 520. In some embodiments, the tip portion 522 of the fourth plurality of wedges 520 extends in the same general direction from the bottom portion 524 of the fourth plurality of wedges 520 as the tip portion 422 of the third plurality of wedges 420 extends from the bottom portion 424 of the third plurality of wedges 420 and/or the tip portion 322 of the second plurality of wedges 320 extends from the bottom portion 324 of the second plurality of wedges 320 and/or the tip portion 222 of the first plurality of wedges 220 extends from the bottom portion 224 of the first plurality of wedges 220. Other configurations are also contemplated.

FIG. 10 illustrates different sizes of the plurality of foam layers 110 and/or the first disk-shaped piece of foam 200, the second disk-shaped piece of foam 300, the third disk-shaped piece of foam 400, and the fourth disk-shaped piece of foam 500 that may be used and/or may be present in the implant 100. The skilled artisan will recognize that the plurality of foam layers 110 and/or the various disk-shaped pieces of foam described herein may be used in various arrangements and/or combinations to suit and/or achieve design intents. In some embodiments, a system and/or a kit may comprise different sizes of the plurality of foam layers 110 and/or the first disk-shaped piece of foam 200, the second disk-shaped piece of foam 300, the third disk-shaped piece of foam 400, and the fourth disk-shaped piece of foam 500 such that the practitioner may choose individual elements to make the implant 100 according to the size of the patient’s left atrial appendage. As such, FIG. 10 may be considered to illustrates selected aspects of the system or kit.

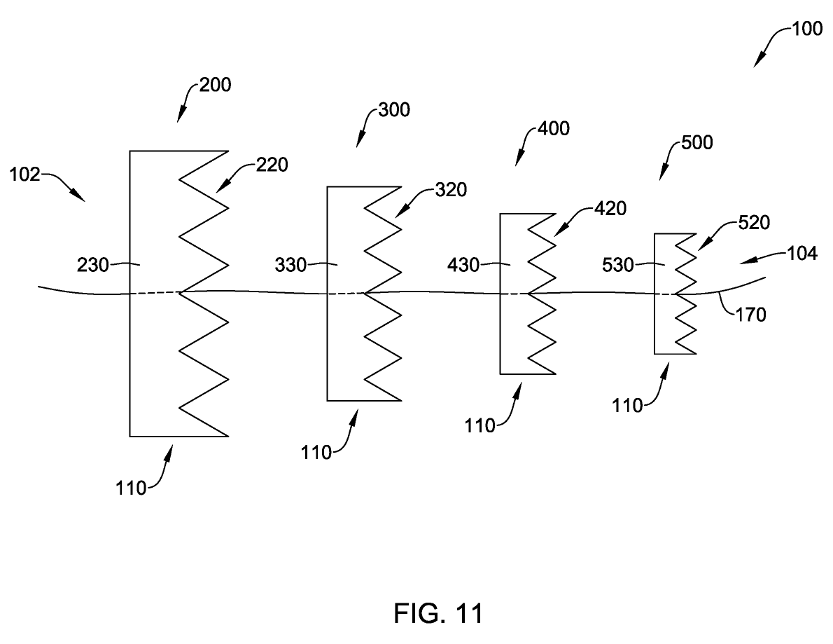

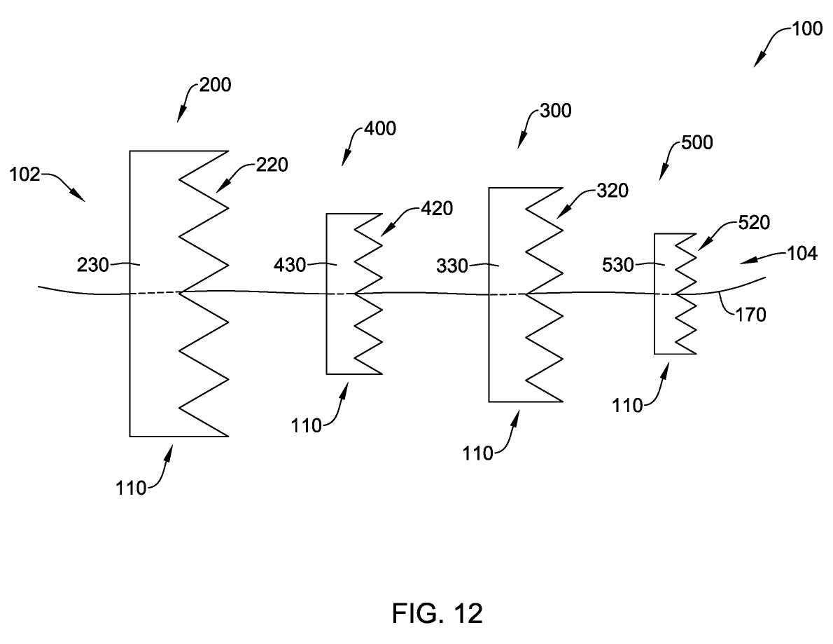

In some embodiments, the plurality of foam layers 110 may be attached together with a suture 170, as seen in FIGS. 11-12. In some embodiments, the plurality of foam layers 110 may be attached together via adhesive bonding. Other configurations, including combinations thereof, are also contemplated. In some embodiments, a system or kit according to the disclosure may comprise the suture 170 and/or an adhesive and/or bonding agent.

In some embodiments, each foam layer of the plurality of foam layers 110 may have an outermost radial extent that reduces in a direction from the first axial end 102 of the implant 100 to the second axial end 104 of the implant 100. In some embodiments, each successive foam layer of the plurality of foam layers 110 from the first axial end 102 of the implant 100 to the second axial end 104 of the implant 100 may have a smaller outermost radial extent than the previous foam layer of the plurality of foam layers 110. In some embodiments, every successive foam layer of the plurality of foam layers 110 from the first axial end 102 of the implant 100 to the second axial end 104 of the implant 100 may have a smaller outermost radial extent than the previous foam layer of the plurality of foam layers 110.

In some embodiments, the first disk-shaped piece of foam 200 may have a first outermost radial extent, the second disk-shaped piece of foam 300 may have a second outermost radial extent less than the first outermost radial extent, the third disk-shaped piece of foam 400 may have a third outermost radial extent less than the second outermost radial extent, and the fourth disk-shaped piece of foam 500 may have a fourth outermost radial extent less than the third outermost radial extent, as seen in FIGS. 10-11. In some embodiments, the first disk-shaped piece of foam 200 may have a first outermost radial extent, the second disk-shaped piece of foam 300 may have a second outermost radial extent less than the first outermost radial extent, the third disk-shaped piece of foam 400 may have a third outermost radial extent less than the first outermost radial extent and greater than the second outermost radial extent, and the fourth disk-shaped piece of foam 500 may have a fourth outermost radial extent less than the third outermost radial extent, the second outermost radial extent, and the first outermost radial extent, as seen in FIG. 12. Other configurations and/or arrangements are also contemplated.

In some embodiments, the method of making the implant 100 for occluding the left atrial appendage may comprise attaching the cap layer of foam 160 (e.g., FIGS. 2-3) to the first disk-shaped piece of foam 200 (e.g., FIG. 11) opposite the second disk-shaped piece of foam 300 (e.g., FIG. 11). In some embodiments, the method of making the implant 100 for occluding the left atrial appendage may comprise attaching the cap layer of foam 160 (e.g., FIGS. 2-3) to and/or at the first axial end 102 of the implant 100, as seen in FIG. 2.

In some embodiments, the method of making the implant 100 for occluding the left atrial appendage may comprise attaching the cap layer of foam 160 (e.g., FIGS. 2-3) to the fourth disk-shaped piece of foam 500 (e.g., FIG. 11) opposite the third disk-shaped piece of foam 400 (e.g., FIG. 11). In some embodiments, the method of making the implant 100 for occluding the left atrial appendage may comprise attaching the cap layer of foam 160 (e.g., FIGS. 2-3) to and/or at the second axial end 104 of the implant 100, as seen in FIG. 3.

In some embodiments, an outermost radial extent of the cap layer of foam 160 may be less than an outermost radial extent (e.g., the first outermost radial extent) of the first disk-shaped piece of foam 200 and/or the first plurality of wedges 220. In some alternative configurations, an outermost radial extent of the cap layer of foam 160 may be greater than an outermost radial extent (e.g., the first outermost radial extent) of the first disk-shaped piece of foam 200 and/or the first plurality of wedges 220. In some further alternative configurations, an outermost radial extent of the cap layer of foam 160 may be similar to and/or the same as an outermost radial extent (e.g., the first outermost radial extent) of the first disk-shaped piece of foam 200 and/or the first plurality of wedges 220.

FIG. 13 schematically illustrates one configuration of the implant 100 disposed within a left atrial appendage 10. It shall be understood that the left atrial appendage 10 of FIG. 13 is merely exemplary, and other geometries, shapes, sizes, etc. for the left atrial appendage 10 are common and may vary from patient to patient. The left atrial appendage 10 may be formed as a small pouch or extension attached to and extending from the left atrium of a patient’s heart.

The left atrial appendage 10 may include a longitudinal axis arranged along a depth of a main body 20 of the left atrial appendage 10. The main body 20 may include a side wall 22 and an ostium 30 forming a proximal mouth. In some embodiments, a lateral extent of the main body 20 and/or the ostium 30 may be smaller or less than a depth of the main body 20 along the longitudinal axis, or a depth of the main body 20 may be greater than a lateral extent of the main body 20 and/or the ostium 30. In some embodiments, the left atrial appendage 10 may narrow quickly along the depth of the main body 20 or the left atrial appendage 10 may maintain a generally constant lateral extent along a majority of the depth of the main body 20.

In some embodiments, the left atrial appendage 10 may include a distalmost region 12 formed or arranged as a tail-like element associated with a distal portion of the main body 20. In some embodiments, the distalmost region 12 may protrude radially or laterally away from the longitudinal axis and/or the main body 20.

As seen in FIG. 13, the implant 100 may be deployed within the left atrial appendage 10 proximate the ostium 30. In some embodiments, the cap layer of foam 160 may be disposed within the ostium 30 and/or may fill and/or block the proximal mouth of the ostium 30. In some embodiments, the cap layer of foam 160 may define a proximalmost surface of the implant 100 and/or may be configured to face outward from the left atrial appendage 10 and/or to face toward the left atrium. In some embodiments, the proximalmost surface of the implant 100 may comprise and/or may be coated with a therapeutic agent. In some embodiments, other surfaces of the implant 100 (e.g., surfaces other than the proximalmost surface) may comprise and/or may be coated with a therapeutic agent. In some embodiments, the proximalmost surface of the implant 100 and the other surfaces of the implant 100 may comprise and/or may be coated with different therapeutic agents. Other configurations are also contemplated.

In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured to shift from a first configuration (e.g., a compressed configuration) to a second configuration (e.g., an expanded configuration). In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be constrained in the first configuration (e.g., the compressed configuration) by a delivery device. In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured to shift from the first configuration (e.g., the compressed configuration) to the second configuration (e.g., the expanded configuration) when unconstrained.

In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured to shift from the first configuration (e.g., the compressed configuration) to the second configuration (e.g., the expanded configuration) in vivo. In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured to adapt and conform to the left atrial appendage 10 (e.g., FIG. 13) and/or surrounding anatomy when shifting from the first configuration (e.g., the collapsed configuration) toward and/or to the second configuration (e.g., the expanded configuration) in vivo.

In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured to shift from the first configuration (e.g., the compressed configuration) to the second configuration (e.g., the expanded configuration) when exposed to a stimulus, such as a preselected temperature or a fluid. In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be formed from a shape memory material. In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be formed from different shape memory materials. Other configurations are also contemplated.

In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may have a first overall volume in the first configuration (e.g., the compressed configuration). In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may have a second overall volume in the second configuration (e.g., the expanded configuration) different from the first overall volume. In some embodiments, the second overall volume may be about 10% different, about 20% different, about 30% different, about 40% different, about 50% different, about 75% different, about 100% different, about 125% different, about 150% different, about 200% different, etc. from the first overall volume. In at least some embodiments, the second overall volume may be greater than the first overall volume.

In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may comprise and/or may be formed from a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed or delivery size. In some examples, the collapsibility ratio of the shape memory polymer and/or the shape memory foam may be at least 5 times, at least 7 times, at least 8 times, at least 9 times, at least 10 times, at least 12 times, or more. Other configurations are also contemplated. In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured as open celled foam.

In some embodiments, the plurality of foam layers 110, and/or the first disk-shaped piece of foam 200, the second disk-shaped piece of foam 300, the third disk-shaped piece of foam 400, and the fourth disk-shaped piece of foam 500, and/or the shape memory foam may be formed from a biocompatible material. In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be non-biodegradable and/or non-bioabsorbable. In some alternative embodiments, the plurality of foam layers 110, and/or the first disk-shaped piece of foam 200, the second disk-shaped piece of foam 300, the third disk-shaped piece of foam 400, and the fourth disk-shaped piece of foam 500, may be biodegradable and/or bioabsorbable over time. In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured to promote endothelization and/or tissue ingrowth. In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may include a coating, a material, and/or a component that promotes endothelization and/or tissue ingrowth. Other configurations are also contemplated.

In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured to prevent thrombus formation. In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may include an anti-thrombus agent(s) and/or medicament(s). In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured to absorb blood and/or bodily fluid(s). In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured to trap thrombus. In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be configured to promote tissue ingrowth and/or endothelization. Other configurations are also contemplated.

In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be unconstrained by outside forces and/or structure(s) in the first configuration (e.g., the collapsed configuration). In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may be self-maintained in the first configuration (e.g., the collapsed configuration) by shape memory properties. Other configurations are also contemplated.

In some embodiments, the plurality of foam layers 110, and/or the first disk-shaped piece of foam 200, the second disk-shaped piece of foam 300, the third disk-shaped piece of foam 400, and the fourth disk-shaped piece of foam 500, may optionally comprise a radiopaque marker coupled thereto and/or embedded therein. In some embodiments, the radiopaque marker may comprise a radiopaque substance or a radiopaque material disposed within the plurality of foam layers 110 and/or the first disk-shaped piece of foam 200, the second disk-shaped piece of foam 300, the third disk-shaped piece of foam 400, and the fourth disk-shaped piece of foam 500 (e.g., one or more of the plurality of foam layers 110 and/or the first disk-shaped piece of foam 200, the second disk-shaped piece of foam 300, the third disk-shaped piece of foam 400, and the fourth disk-shaped piece of foam 500 may be doped with and/or may include the radiopaque substance or the radiopaque material).

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the implant, the plurality of foam layers, the disk-shaped pieces of foam, the cap layer, the suture(s), etc. and/or elements or components thereof.

The foam layers may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. Suitable transition temperatures may be, for example, at or below about 37 °C (about 98.6 °F), which allows the shape memory foam to assume an initial configuration prior to and during delivery through a delivery catheter or other delivery device, and an expanded configuration for occlusion after delivery and release within the left atrial appendage, allowing the foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the foam in the expanded configuration will be from about 10 kg/m3 (about 0.62 lb/ft3) to about 1000 kg/m3 (about 62.31 lb/ft3), including from about 10 kg/m3 to about 500 kg/m3 (about 31.2 lb/ft3) including from about 10 kg/m3 to about 200 kg/m3 (about 12.5 lb/ft3), including from about 20 kg/m3 to about 100 kg/m3 (about 6.2 lb/ft3).

Generally, the material for constructing the foam layers is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the foam layers may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the foam layers may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerization of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha,omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the foam layers may include thermoset polyurethanes that include oxidatively susceptible linkages in the soft segment, including but not limited to tertiary amines and polyethers. The foam layers may optionally include hydrolytically degradable soft segment components such as polycaprolactone, esters, and others. In some cases, the polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni-Co-Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. An implant for occluding a left atrial appendage, comprising:
a plurality of foam layers attached together;
wherein each foam layer of the plurality of foam layers comprises a plurality of wedges monolithically formed with a base portion.

2. The implant of claim 1, wherein the plurality of wedges of each foam layer are interdigitated with the plurality of wedges of an immediately adjacent foam layer.

3. The implant of claim 1, wherein at least one foam layer of the plurality of foam layers has an outermost radial extent that is different from at least one other foam layer of the plurality of foam layers.

4. The implant of claim 3, wherein each foam layer of the plurality of foam layers has a different outermost radial extent.

5. The implant of claim 1, wherein at least one foam layer of the plurality of foam layers has a density that is different from at least one other foam layer of the plurality of foam layers.

6. The implant of claim 1, wherein at least one wedge of the plurality of wedges within a selected foam layer of the plurality of foam layers has a density that is different from at least one other wedge of the plurality of wedges within the selected foam layer.

7. The implant of claim 1, wherein at least one foam layer of the plurality of foam layers is configured as an anchoring layer configured to exert an anchoring force radially outward against a side wall of the left atrial appendage.

8. The implant of claim 1, wherein the plurality of foam layers comprises a first anchoring layer configured to exert a first anchoring force radially outward against a side wall of the left atrial appendage and a second anchoring layer configured to exert a second anchoring force radially outward against the side wall of the left atrial appendage, wherein the second anchoring layer is axially spaced apart from the first anchoring layer.

9. The implant of claim 8, wherein the plurality of foam layers comprises a low-density fill layer disposed between the first anchoring layer and the second anchoring layer.

10. An implant for occluding a left atrial appendage, comprising:
a plurality of foam layers attached together;
wherein each foam layer of the plurality of foam layers comprises a plurality of wedges monolithically formed with a base portion; and
a cap layer of foam attached at an axial end of the plurality of foam layers attached together;
wherein the cap layer of foam is disk-shaped and is devoid of wedges.

11. The implant of claim 10, wherein the cap layer of foam has an outermost radial extent that is less than a maximum outermost radial extent of the plurality of foam layers.

12. The implant of claim 11, wherein the cap layer of foam is disposed immediately adjacent a foam layer of the plurality of foam layers that has the maximum outermost radial extent.

13. The implant of claim 11, wherein the cap layer of foam is disposed immediately adjacent a foam layer of the plurality of foam layers having a minimum outermost radial extent of the plurality of foam layers.

14. A method of making an implant for occluding a left atrial appendage, comprising:
cutting a first disk-shaped piece of foam partially through a thickness of the first disk-shaped piece of foam to define a first plurality of wedges monolithically formed with a first base portion;
securing the first base portion in a contracted configuration to flare the first plurality of wedges apart from each other;
cutting a second disk-shaped piece of foam partially through a thickness of the second disk-shaped piece of foam to define a second plurality of wedges monolithically formed with a second base portion;
securing the second base portion in a contracted configuration to flare the second plurality of wedges apart from each other; and
attaching the first base portion to the second base portion such that the first plurality of wedges is interdigitated with the second plurality of wedges.

15. The method of claim 14, wherein the first base portion is 25% to 50% of the thickness of the first disk-shaped piece of foam.

16. The method of claim 14, wherein after attaching the first base portion to the second base portion, each wedge of the first plurality of wedges defines a tip portion and a bottom portion, and each wedge of the second plurality of wedges defines a tip portion and a bottom portion; and
wherein the tip portion of the first plurality of wedges extends in a first direction from the bottom portion of the first plurality of wedges, and the tip portion of the second plurality of wedges extends in the first direction from the bottom portion of the second plurality of wedges.

17. The method of claim 14, wherein cutting the first disk-shaped piece of foam partially through the thickness of the first disk-shaped piece of foam comprises cutting downward into a top surface of the first disk-shaped piece of foam;
wherein after securing the first base portion in the contracted configuration, the top surface of the first disk-shaped piece of foam faces radially outwardly.

18. The method of claim 14, wherein further comprising:
attaching a cap layer of foam to the first disk-shaped piece of foam opposite the second disk-shaped piece of foam, wherein an outermost radial extent of the cap layer of foam is less than an outermost radial extent of the first disk-shaped piece of foam.

19. The method of claim 14, further comprising:
cutting a third disk-shaped piece of foam partially through a thickness of the third disk-shaped piece of foam to define a third plurality of wedges monolithically formed with a third base portion;
securing the third base portion in a contracted configuration to flare the third plurality of wedges apart from each other; and
attaching the third base portion to the second base portion such that the second plurality of wedges is interdigitated with the third plurality of wedges.

20. The method of claim 19, further comprising:
cutting a fourth disk-shaped piece of foam partially through a thickness of the fourth disk-shaped piece of foam to define a fourth plurality of wedges monolithically formed with a fourth base portion;
securing the fourth base portion in a contracted configuration to flare the fourth plurality of wedges apart from each other; and
attaching the fourth base portion to the third base portion such that the third plurality of wedges is interdigitated with the fourth plurality of wedges.
